SEQUENCE LISTING (11 sequences)

SEQ ID NO: 1 - protein, 424 aa, Artificial Sequence
  SPOCK2 protein
MRAPGCGRLVLPLLLLAAAALAEGDAKGLKEGETPGNFMEDEQWLSSISQYSGKIKHWNRFRDEVEDDYIKSWEDNQQGDEALDTTKDPCQKVKCSRHKVCIAQGYQRAMCISRKKLEHRIKQPTVKLHGNKDSICKPCHMAQLASVCGSDGHTYSSVCKLEQQACLSSKQLAVRCEGPCPCPTEQAATSTADGKPETCTGQDLADLGDRLRDWFQLLHENSKQNGSASSVAGPASGLDKSLGASCKDSIGWMFSKLDTSADLFLDQTELAAINLDKYEVCIRPFFNSCDTYKDGRVSTAEWCFCFWREKPPCLAELERIQIQEAAKKKPGIFIPSCDEDGYYRKMQCDQSSGDCWCVDQLGLELTGTRTHGSPDCDDIVGFSGDFGSGVGWEDEEEKETEEAGEEAEEEEGEAGEADDGGYIW

SEQ ID NO: 2 - protein, 424 aa, Artificial Sequence
  SPOCK2 variant(N225D)
MRAPGCGRLVLPLLLLAAAALAEGDAKGLKEGETPGNFMEDEQWLSSISQYSGKIKHWNRFRDEVEDDYIKSWEDNQQGDEALDTTKDPCQKVKCSRHKVCIAQGYQRAMCISRKKLEHRIKQPTVKLHGNKDSICKPCHMAQLASVCGSDGHTYSSVCKLEQQACLSSKQLAVRCEGPCPCPTEQAATSTADGKPETCTGQDLADLGDRLRDWFQLLHENSKQDGSASSVAGPASGLDKSLGASCKDSIGWMFSKLDTSADLFLDQTELAAINLDKYEVCIRPFFNSCDTYKDGRVSTAEWCFCFWREKPPCLAELERIQIQEAAKKKPGIFIPSCDEDGYYRKMQCDQSSGDCWCVDQLGLELTGTRTHGSPDCDDIVGFSGDFGSGVGWEDEEEKETEEAGEEAEEEEGEAGEADDGGYIW

SEQ ID NO: 3 - DNA, 1275 bp, Artificial Sequence
  SPOCK2 protein
atgcgcgccccgggctgcgggcggctggtgctgccgctgctgctcctggccgcggcagccctggccgaaggcgacgccaaggggctcaaggagggcgagacccccggcaatttcatggaggacgagcaatggctgtcgtccatctcgcagtacagcggcaagatcaagcactggaaccgcttccgagacgaagtggaggatgactatatcaagagctgggaggacaatcagcaaggagatgaagccctggataccaccaaggacccctgccagaaggtgaagtgcagccgccacaaggtgtgcattgcccagggctaccagcgggccatgtgcatcagtcgcaagaagctggagcacaggatcaagcagccgaccgtgaaactccatggaaacaaagactccatctgcaagccctgccacatggcccagcttgcctctgtctgcggctcagatggccacacttacagctctgtgtgtaagctggagcaacaggcgtgcctgagcagcaagcagctggcggtgcgatgcgagggcccctgcccctgccccacggagcaggctgccacctccaccgccgatggcaaaccagagacttgcaccggtcaggacctggctgacctgggagatcggctgcgggactggttccagctccttcatgagaactccaagcagaatggctcagccagcagtgtagccggcccggccagcgggctggacaagagcctgggggccagctgcaaggactccattggctggatgttctccaagctggacaccagtgctgacctcttcctggaccagacggagctggccgccatcaacctggacaagtacgaggtctgcatccgtcccttcttcaactcctgtgacacctacaaggatggccgggtctctactgctgagtggtgcttctgcttctggagggagaagcccccctgcctggcagagctggagcgcatccagatccaggaggccgccaagaagaagccaggcatcttcatcccgagctgcgacgaggatggctactaccggaagatgcagtgtgaccagagcagcggtgactgctggtgtgtggaccagctgggcctggagctgactggcacgcgcacgcatgggagccccgactgcgatgacatcgtgggcttctcgggggactttggaagcggtgtcggctgggaggatgaggaggagaaggagacggaggaagcaggcgaggaggccgaggaggaggagggcgaggcaggcgaggctgacgacgggggctacatctggtag

SEQ ID NO: 4 - DNA, 1275 bp, Artificial Sequence
  SPOCK2 variant (N225D)
atgcgcgccccgggctgcgggcggctggtgctgccgctgctgctcctggccgcggcagccctggccgaaggcgacgccaaggggctcaaggagggcgagacccccggcaatttcatggaggacgagcaatggctgtcgtccatctcgcagtacagcggcaagatcaagcactggaaccgcttccgagacgaagtggaggatgactatatcaagagctgggaggacaatcagcaaggagatgaagccctggataccaccaaggacccctgccagaaggtgaagtgcagccgccacaaggtgtgcattgcccagggctaccagcgggccatgtgcatcagtcgcaagaagctggagcacaggatcaagcagccgaccgtgaaactccatggaaacaaagactccatctgcaagccctgccacatggcccagcttgcctctgtctgcggctcagatggccacacttacagctctgtgtgtaagctggagcaacaggcgtgcctgagcagcaagcagctggcggtgcgatgcgagggcccctgcccctgccccacggagcaggctgccacctccaccgccgatggcaaaccagagacttgcaccggtcaggacctggctgacctgggagatcggctgcgggactggttccagctccttcatgagaactccaagcaggatggctcagccagcagtgtagccggcccggccagcgggctggacaagagcctgggggccagctgcaaggactccattggctggatgttctccaagctggacaccagtgctgacctcttcctggaccagacggagctggccgccatcaacctggacaagtacgaggtctgcatccgtcccttcttcaactcctgtgacacctacaaggatggccgggtctctactgctgagtggtgcttctgcttctggagggagaagcccccctgcctggcagagctggagcgcatccagatccaggaggccgccaagaagaagccaggcatcttcatcccgagctgcgacgaggatggctactaccggaagatgcagtgtgaccagagcagcggtgactgctggtgtgtggaccagctgggcctggagctgactggcacgcgcacgcatgggagccccgactgcgatgacatcgtgggcttctcgggggactttggaagcggtgtcggctgggaggatgaggaggagaaggagacggaggaagcaggcgaggaggccgaggaggaggagggcgaggcaggcgaggctgacgacgggggctacatctggtag

SEQ ID NO: 5 - RNA, 19 nt, Artificial Sequence
  SPOCK2 siRNA #1
gagacgaaguggaggauga

SEQ ID NO: 6 - RNA, 21 nt, Artificial Sequence
  control siRNA
uucuccgaacgugucacguuu

SEQ ID NO: 7 - DNA, 20 bp, Artificial Sequence
  SPOCK2_F primer
gtgactgctggtgtgtggac

SEQ ID NO: 8 - DNA, 21 bp, Artificial Sequence
  SPOCK2_R primer
cttcctccgtctccttctcct

SEQ ID NO: 9 - DNA, 20 bp, Artificial Sequence
  HA_F primer
ttgctaaaacccggagacac

SEQ ID NO: 10 - DNA, 20 bp, Artificial Sequence
  HA_R primer
cctgacgtattttgggcact

SEQ ID NO: 11 - RNA, 1414 nt, Artificial Sequence
  NS GFP (-)RNA
cauggugagcaagggcgaggagcuguucaccgggguggugcccauccuggucgagcuggacggcgacguaaacggccacaaguucagcguguccggcgagggcgagggcgaugccaccuacggcaagcugacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuucaaggacgacggcaacuacaagacccgcgccgaggugaaguucgagggcgacacccuggugaaccgcaucgagcugaagggcaucgacuucaaggaggacggcaacauccuggggcacaagcuggaguacaacuacaacagccacaacgucuauaucauggccgacaagcagaagaacggcaucaaggugaacuucaagauccgccacaacaucgaggacggcagcgugcagcucgccgaccacuaccagcagaacacccccaucggcgacggccccgugcugcugcccgacaaccacuaccugagcacccaguccgcccugagcaaagaccccaacgagaagcgcgaucacaugguccugcuggaguucgugaccgccgccgggaucacucucggcauggacgagcuguacaaggaauugauccaaacacugugucaagcuuucagguagauugcuuucuuuggcauguccgcaaaagaguugcagaccaagaacuaggugaugccccauuccuugaucggcuucgccgagaucagaagucccuaagaggaagaggcagcacucuuggucuggacaucgaaacagccacccgugcuggaaagcaaauaguggagcggauucugaaaggaagaaucugaugaggcacucaaaaugaccauggccucuguaccugcaucgcgcuaccuaacugacaugacucuugaggaaaugucaaggcacugguucaugcucaugcccaagcagaaaguggcaggcccucuuuguaucagaauggaccaggcgaucauggauaagaacaucauacugaaagcgaacuucagugugauuuuugaccggcuggagacucuaauauuacuaagggccuucaccgaagaggggacaauuguuggcgaaauuucaccacugcccucucuuccaggacauacugaugaggaugucaaaaaugcaguugggguccucaucggaggacuugaauggaauaauaacacaguucgagucucugaaacucuacagagauucgcuuggagaagcaguaaugagaaugggagaccuccacucacuccaaaacagaaacgagaaauggcgggaacaauuaggucagaaguuuga

(19) United States
(12) Patent Application Publication
(10) Pub. No.: US 2020/0038502 A1
(43) Pub. Date: Feb. 6, 2020

(54) PHARMACEUTICAL COMPOSITION PREVENTING OR TREATING AN INFLUENZA VIRAL INFECTIOUS DISEASE

(71) Applicant: POSTECH ACADEMY-INDUSTRY FOUNDATION, Pohang-si (KR)

(72) Inventors: Joo-Yeon YOO, Pohang-si (KR); Na Rae Ahn, Pohang-si (KR)

(21) Appl. No.: 16/584,777

(22) Filed: Sep. 26, 2019

Related U.S. Application Data
(63) Continuation of application No. 16/068,407, filed on Jul. 6, 2018, now abandoned.

(30) Foreign Application Priority Data
Feb. 4, 2016 (KR) 10-2016-0014478

(51) Int. Cl. A61K 39/145; A61K 48/00; A61K 39/12; A61P 31/16

(52) U.S. Cl. CPC A61K 39/145; A61K 48/00; A61K 2039/525; A61P 31/16; C12N 2760/16134; A61K 39/12

(57) ABSTRACT

The present invention provides a vaccine composition and pharmaceutical composition for preventing or treating a viral infectious disease comprising one or more selected from the group consisting of SPOCK2 protein and a gene encoding the SPOCK2 protein as an active ingredient, a method of preparing the same, and prevention and treatment method.

### TECHNICAL FIELD

The present invention relates to a pharmaceutical composition for preventing or treating an influenza viral infectious disease and a method for treating or preventing an influenza viral infectious disease comprising a step of administering a pharmaceutically effective dose of the composition, and more specifically, relates to a pharmaceutical composition comprising one or more selected from the group consisting of SPOCK2 protein, a gene encoding the SPOCK2 protein, a vector comprising the gene and a cell comprising the vector as an active ingredient and a method for using the same.

### BACKGROUND ART

An infection disease is an infection caused by microscopic organisms such as bacteria, viruses, fungi, etc., and it is directly or indirectly infectious. The World Health Organization categorizes the infection disease as one of the top 10 causes of death, and the ratio is high especially in low-income countries.

Influenza viruses are infected through the respiratory tract and it is reported that 5-10% adults and 20-30% of children are infected every year in worldwide. The symptoms by the infection of influenza viruses range from mere high fever, cough, stomachache and muscle ache to death severely, and 250,000 to 500,000 people die by infection every year. The influenza viruses have 8 segmented single stranded RNAs as their genome, and consist of the structure enveloped by envelope proteins. The infection of influenza viruses is initiated by the binding of a hemaglutinin (HA) protein expressed on the surface of viruses and a sialic acid receptor expressed on the surface of host cell. After entering in the cell, they are replicated and assembled using host cell proteins and released to the outside of the cell to proliferate. It has been reported that the major structural protein of influenza viruses, NP protein, is involved in transcription and replication. It has been known that the NP protein encapsulating the virus RNA comprises a nuclear translocation sequence to play a role in not only delivering the virus RNA into a nucleus but also stabilizing the transcribed and replicated RNA.

The current influenza virus therapeutic agents mainly target virus proteins and have the effect of inhibiting the life cycle (invasion, replication and release) of viruses. The neuraminidase (NA) of viruses is a glycoprotein having an enzymatic activity of sialidase, and Tamiflu which is a representative therapeutic agent of influenza viruses targets the NA protein and inhibits the release of viruses to inhibit the proliferation of viruses. The NA protein of viruses is involved in the intracellular invasion by combining to the sialic acid receptor on the host cell surface. Inhibitors controlling the attachment and invasion of viruses by inhibiting the function of HA targeting the sialidase of host cell or Neu5Ac have been developed and researched.

### DISCLOSURE

### Technical Problem

The present invention is intended to provide a DNA vaccine composition for preventing or treating a viral infectious disease, comprising a gene encoding SPOCK2 protein as an active ingredient.

The present invention is also intended to provide a DNA vaccine composition for preventing or treating an influenza viral infectious disease, comprising a SPOCK2 protein as an active ingredient.

The present invention is also intended to provide a method for preparing a DNA vaccine composition for preventing or treating, comprising a step of modifying a codon of a gene encoding SPOCK2 protein so that the SPOCK2 protein is overexpressed in a cell; and a step of introducing the modified gene into a vector.

The present invention is also intended to provide a method for preventing or treating an influenza virus disease comprising a step of administering a pharmaceutically effective dose of the composition.

### Technical Solution

The present inventors have developed a DNA vaccine composition for preventing or treating a viral infectious disease, comprising a gene encoding SPOCK2 protein, by confirming that the SPOCK2 protein or its glycosylation plays an important role in the proliferation of viruses, in particular, influenza A virus, and its control of infection, in order to achieve the above purposes.

One embodiment of the present invention is a DNA vaccine composition for preventing or treating a viral infectious disease, comprising a gene encoding SPOCK2 protein.

Another embodiment of the present invention is a DNA vaccine composition for preventing or treating an influenza viral infectious disease, comprising SPOCK2 protein as an active ingredient.

Other embodiment of the present invention is a method for preparing a composition for preventing or treating a virus DNA vaccine, comprising a step of modifying a gene codon encoding SPOCK2 protein so that SPOCK2 protein is overexpressed in a cell; and a step of introducing the modified gene into a vector.

Other embodiment of the present invention relates to a method for preventing or treating a virus disease comprising a step of administering a pharmaceutically effective dose of the composition.

According to the DNA vaccine composition for preventing or treating a viral infectious disease of the present invention, the proliferation of virus is inhibited, and it may be used for virus prevention or infection treatment. The inhibition of influenza virus proliferation may be achieved by one or more selected from the group consisting of inhibition of intracellular invasion of virus, inhibition of expression of nucleoprotein (NP) protein of virus, and inhibition of expression of hemagglutinin (HA) protein of virus.

Hereinafter, the present invention will be described in more detail.

One embodiment of the present invention relates to a DNA vaccine composition for preventing or treating a viral infectious disease, comprising a gene encoding SPOCK2 protein or SPOCK2 protein. In addition, the gene may be provided in one or more forms selected from the group consisting of a form of polynucleotide encoding the SPOCK2 protein, a vector comprising the polynucleotide, and a cell comprising the vector.

SPOCK2 protein is a glycoprotein consisting of 422 amino acids. It consists of signal peptide commonly shown in a secretion protein is present in the N-terminal, FS domain, EC domain binding with calcium, and Glu domain. It is known that the SPOCK2 protein produced in the cell undergoes a glycosylation process through ER, Golgi which are intracellular organelles, and is finally secreted to the outside of the cell and mainly located in the extracellular membrane. Conventionally, it is reported that the expression of SPOCK2 is increased in the development process of lung or brain, and it plays a role of controlling their development and maintenance, and it is revealed that it is excessively methylated in colon, prostate, breast cancer, etc., and therefore, it has been used as a biomarker for diagnosing cancer.

SPOCK2 is glycosylated, and may comprise N-linked glycosylation site in the 225th asparagine residue and glycosaminoglycan attachment sites in the 383th and 388th serine residues. In the SPOCK2, the glycosylation in the positions of at least one amino acid selected from the group consisting of the 225th, 383th and 388th may be occurred, and for example, the molecular weight of glycosylated SPOCK2 may be preferably 55 to 170 kDa, more preferably 55 to 100 kDa.

For example, when the glycosylation is inhibited by substituting the 225th asparagine residue of SPOCK2 protein with an aspartic acid residue, the effect of inhibiting the proliferation of influenza virus by SPOCK2 protein is removed, and the amount of influenza virus released to the outside of the cell is also increased. This means that the glycosylation phenomenon of SPOCK2 protein is involved in the effect of inhibiting the virus proliferation.

The gene encoding SPOCK2 protein may be characterized by that the codon is modified so that the SPOCK2 protein is overexpressed in the cell, and in particular, may comprise a variant of SPOCK2 protein capable of glycosylation. This results from that the glycosylation of SPOCK2 protein plays an important role in the control of virus infection and proliferation.

Preferably, the gene encoding SPOCK2 protein may be a nucleotide sequence coding SPOCK2 protein consisting of an amino acid sequence of SEQ ID NO: 1. The gene may be a nucleotide sequence of SEQ ID NO: 3.

It was confirmed by the inhibition of expression of nucleoprotein (NP) protein or hemagglutinin (HA) protein of virus that the proliferation of influenza virus is inhibited, and specifically the intracellular invasion of virus may be inhibited and the virus proliferation is inhibited, when the SPOCK2 protein is overexpressed.

The gene encoding SPOCK2 protein may be provided in a form contained in a vector, and preferably the vector may be a virus or non-virus vector. In particular, the virus vector may be one or more selected from the group consisting of adenovirus, adeno-associated virus, helper-dependent adenovirus and retrovirus vectors. Specifically, the vector may have a cleavage map of FIG. 1. In addition, the non-virus vector may be a plasmid, liposome, etc.

The gene encoding SPOCK2 protein may be provided in a form of cell transformed by a vector containing the gene.

The composition may inhibit the proliferation of virus to prevent or treat a viral infectious disease. The method for inhibiting the proliferation of virus is not limited to methods for inhibiting the life cycle of virus, and preferably may be one or more selected from the group consisting of inhibition of intracellular invasion of virus; inhibition of expression of nucleoprotein (NP) protein of virus; and inhibition of expression of hemagglutinin (HA) protein of virus.

The method for inhibiting the proliferation of virus may be performed in vivo or in vitro.

The virus of the present invention is an influenza virus, and more preferably may be an influenza A virus. The viral infectious disease means a disease occurred by the virus infection. Preferably, it may be one or more selected from the group consisting of influenza, Reye syndrome, lung disease and blood cell disease, caused by an influenza virus, and it may be characterized by that the influenza accompanies high fever, cough, stomachache, and the Reye syndrome induces, and the lung disease shows symptoms similar to asthma, and the blood cell disease occurs dyspnea

Other embodiment of the present invention may comprise a formulation for preventing or treating a virus infection of mammal animals, comprising the DNA vaccine composition.

The formulation may further comprise an immuneadjuvant, and any immunoadjuvant known in the art which induces immune and is sate may be preferably used.

The formulation may be formulated as an oral formulation or non-oral formulation, etc. according to common methods respectively to use.

The formulation of the present invention may further contain a pharmaceutically suitable and physiologically acceptable supplemental agent such as carrier, excipient and diluent, etc.

For a specific embodiment to apply the formulation of the present invention to human, the formulation of the present invention may be administered alone, and may be generally administered by being mixed with a pharmaceutical carrier selected with regard to administration means and standard pharmaceutical practice.

The administration dose of the formulation of the present invention may differ from age, weight, gender, administration form, health condition and disease degree of patients, and it may be administered once to several times as divided a day in a certain interval according to the judgment of doctors or pharmacists. For example, the 1 day dosage may be 0.001 to 10000 mg/kg on the basis of content of active ingredient (i.e., SPOCK2 protein, its coding polynucleotide or mixture thereof). The dosage is an example of average case and the dosage may be higher or lower according to the difference of individuals. When the 1 day dosage of pharmaceutical formulation of the present invention is lower than the above administration dose, a significant effect cannot be obtained, and when it is over it, it is uneconomical and also it escapes the range of common dose, and undesirable side effects may be occurred, and therefore the above range is preferable.

Other embodiment of the present invention provides a method for preventing or treating a viral infectious disease comprising a step of administering a pharmaceutically effective dose of the composition.

Other embodiment of the present invention provides a method for preparing an influenza virus DNA vaccine, comprising a step of modifying a codon of gene encoding SPOCK2 protein so that SPOCK2 protein is overexpressed; and a step of introducing the modified gene into a vector.

The details regarding the DNA vaccine composition may be applied to the method for preparing it and the method for preventing or treating a viral infectious disease.

### Effect of the Invention

The pharmaceutical composition of the present invention inhibits the proliferation of virus by one or more selected from the group consisting of SPOCK2 protein and a gene encoding the protein or protein fraction as an active ingredient, and thus it can be usefully used for preventing and/or treating a virus, in particular, influenza A virus (JAY) infection.

### BRIEF DESCRIPTION OF DRAWINGS

FIG. 1 is a drawing showing a cleavage map of vector comprising SPOCK2.

FIG. 2 is a drawing showing the result of measuring GFP signal according to SPOCK2 expression by flow cytometry, after infecting SPOCK2 overexpressing A549 cells with an influenza A virus.

FIG. 3 is a drawing showing the result of measuring the expression of intracellular influenza virus gene (HA) using Real Time-qPCR, after infecting SPOCK2 overexpressing A549 cells with an influenza virus, and the level of SPOCK2-V5 overexpression.

FIG. 4 is a drawing showing the result of analyzing the level of expression of virus protein (NP) of lysates obtained by treatment of lysis buffer by western blot, after infecting SPOCK2 overexpressing A549 cells with an influenza virus.

FIG. 5 is a drawing showing the result of measuring the virus location during the invasion state and attachment state of influenza A virus in SPOCK2 overexpressing A549 cells with a fluorescence microscope, after infection with an influenza virus.

FIG. 6 is a drawing showing the result of measuring the activity of RNA dependent RNA polymerase by SPOCK2 overexpression according to Example 5.

FIG. 7 is a drawing showing the result of measuring the amount of virus released when SPOCK2-V5 is overexpressed according to Example 6.

FIG. 8 is a drawing showing the result of measuring the intracellular location of SPOCK2 according to the virus infection with a fluorescence microscope according to Example 8.

### DETAILED DESCRIPTION OF THE EMBODIMENTS

Hereinafter, the present invention will be described in more detail. However, the following examples are intended to illustrate the present invention, but the present invention is not limited by the following examples.

### Example 1. Preparation of Cell Line Overexpressing SPOCK2

1-1: Preparation of Expression Plasmid

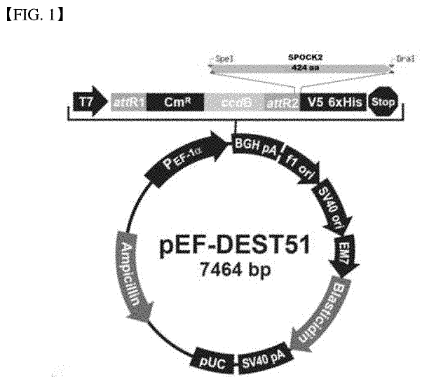

A plasmid expressing SPOCK2 protein was prepared by PCR amplifying a nucleotide sequence of SEQ ID NO: 3 coding SPOCK2 protein (SEQ ID NO: 1) and a nucleotide sequence of SEQ ID NO: 4 coding a protein in which asparagines of 225th sequence of the protein was substituted to aspartic acid (SEQ ID NO: 2) and cloning into pDEST-51 vector. The cleavage map of the plasmid is shown in FIG. 1. The SEQ ID NOs of amino acids and nucleic acids of SPOCK2 protein of SEQ ID NO: 1 and SPOCK2 N225D protein are described in the following Table 1.

TABLE 1

| | SEQ ID NO of Amino | |
|---|---|---|
| Name | acid | SEQ ID NO of Nucleic acid |
| SPOCK2 | 1 | 3 |
| SPOCK2 N225D | 2 | 4 |

1-2: Introduction of Plasmid into Cell Line

After introducing the plasmid prepared in the item 1-1 (SPOCK2-V5) into A549 cell by a liposome injection method, it was cultured for 48 hours in a DMEM media (Welgene) containing 10% fetal bovine serum (hereinafter FBS, Hyclone). In 48 hours, after lysing the A549 cell using a lysis buffer (25 mM Tris-HCl (pH7.4), 150 mM NaCl, 1% Triton X-100, 0.5% deocycholic acid, 0.1% SDS), SPOCK2 overexpressed in the cell was measured by using a V5 (invitrogen, P/N46-0705) antibody. As a result, the overexpression was confirmed by detection of SPOCK2 overexpressed in the cell in which the prepared SPOCK2-V5 plasmid was injected.

### Comparative Example 1. Preparation of Cell Line with Inhibited SPOCK2 Expression

To prepare a cell line in which the expression of SPOCK2 was inhibited as a comparative example, after introducing SPOCK2 siRNA consisting of the sequence of the following Table 2 into A549 cell by a liposome injection method, it was cultured for 48 hours in 10% FBS DMEM media. The control siRNA was used to exclude a non-specific effect of siRNA itself by introduction of siRNA to the cell.

TABLE 2

| | | | SEQ |
|---|---|---|---|
| | | | ID |
| Classification | siRNA | Sequence (5′-3′) | NO |
| Comparative | SPOCK2 siRNA | GAGACGAAGUGGAG | 5 |
| example 1 | #1 | GAUGA | |
| Comparative | control siRNA | UUCUCCGAACGUGUC | 6 |
| example 2 | | ACGUUU | |

### Example 2: Effect of Inhibiting Proliferation of Influenza a Virus

2-1: Influenza a Virus-Infected Cell

After the A549 cell line overexpressing SPOCK2 prepared in Example 1 was in a DMEM media containing no FBS and at the same time, it was infected with GFP-tagged influenza A virus (Prof. adolfo-Garcia Sastre) 10TCID⁵⁰/ml for 24 hours, the cell detached by using trypsin was transferred to a FACS tube, and then immobilized with 4% paraformaldehyde. It was vortexed with FACS buffer (0.5%1-13S in PBS).

2-2: Measurement of Amount of Virus in Cell Through Flow Cytometry

To measure the amount of virus in the cell, the amount of virus in the single cell was quantified by using a flow cytometer. GFP signals were measured by using cytometry.

By the same method, GFP signals were measured by using the flow cytometer for the cell line with inhibited expression of SPOCK2 by introduction of siRNA in the Comparative example 1.

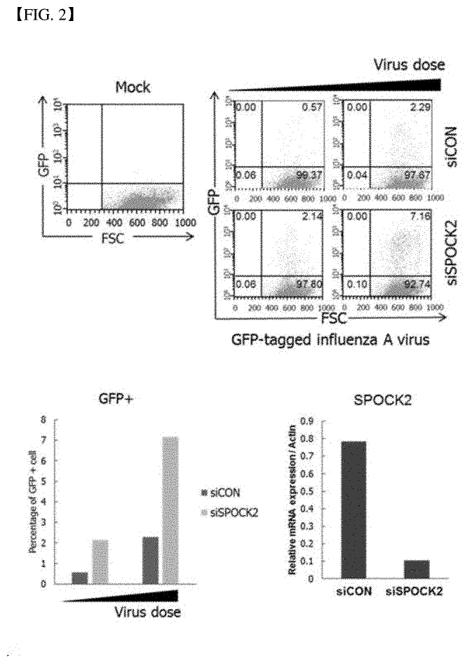

The result of measuring the amount of virus in the A549 cell by GFP and the level of silencing of SPOCK2 by siRNA were shown in FIG. 2. As shown in FIG. 2, it was confirmed that when the SPOCK2 expression was inhibited by siRNA, the influenza virus was increased and the SPOCK2 protein affected the proliferation of influenza virus.

### Example 3. Measurement of Expression of Influenza a Virus Gene

3-1: Influenza a Virus-Infected Cell

V5 was labeled to SPOCK2 according to Example 1, and this was introduced to A549 cell to overexpress SPOCK2, and then an influenza A virus was infected.

3-2: Confirmation of Gene HA Expression

After that, RNA of the cell was extracted by using RNA iso plus (Takara, Japan). To observe the amount of virus in the cell in the RNA level, the expression of virus gene (HA) was measured by using Real Time-qPCR.

Specifically, the cell RNA was extracted by using RNAiso plus (Takara, Japan), and the extracted RNA 0.5 ug was synthesized as cDNA by using Improm-II reverse transcriptase system (Promega, USA) and Random oligomer. For Real time reverse transcriptase polymerase chain reaction, the extracted RNA and primers specifically recognizing each target shown in the following Table 3, SYBR premix Ex-Taq (Takara, Japan) and SOX Rox (Takara, Japan) (primers 5 pmol each, SYBR premix Ex-Taq 2.5 ul, SOX Rox0.2 ul, cDNA 1 ul, DW up to 10 ul) were reacted in One-Step™ Real Time PCR system (Applied Biosystem) (holding stage: 95° C., 15 min cycling stage: [95° C. 15 sec 57° C. 15 sec 72° C. 15 sec] <40 cycles > melting curve stage: [95° C., 5 sec 72° C. 0.5 min 95° C. 15 sec]). The sequences of primers specifically recognizing each target RNA used in the experiment were shown in Table 3.

TABLE 3

| Primer | Sequence (5′-3′) | SEQ ID NO |
|---|---|---|
| SPOCK2_F | GTGACTGCTGGTGTGTGGAC | 7 |
| SPOCK2_R | CTTCCTCCGTCTCCTTCTCCT | 8 |
| HA_F | TTGCTAAAACCCGGAGACAC | 9 |
| HA_R | CCTGACGTATTTTGGGCACT | 10 |

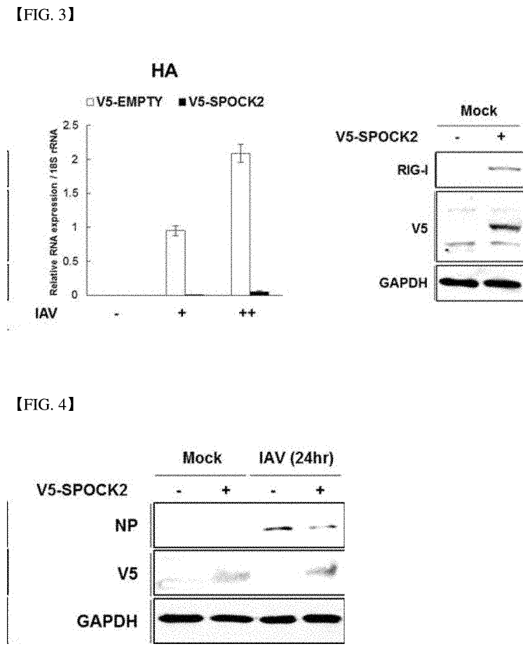

The result of measurement and the level of SPOCK2-V5 overexpression were shown in FIG. 2. As shown in FIG. 3, it was confirmed that when SPOCK2 was overexpressed, the expression of intracellular influenza virus gene (HA) expression was decreased in the RNA level and the proliferation of virus could be inhibited by the SPOCK2 protein.

3-3: Measurement of NP Protein Reduction

After extracting lysates using a lysis buffer in the cell infected in 3-1, the expression of virus protein (NP) was confirmed by a western blot method. Specifically, after isolating lysates that the A549 cell line was lysed with a lysis buffer (25 mM Tris-HCl pH 7.5, 150 mM NaCl, 1% Triton X-100, 0.1% SDS, 0.5% deoxycholate), the expression of each protein was measured by using an antibody specifically recognizing SPOCK2 (Santacruz), NP (Santacruz), and V5 (Invitrogen). The result was shown in FIG. 4.

As can be seen in FIG. 4, it was confirmed that the level of NP protein that was the influenza virus protein was significantly reduced by the expression of SPOCK2 protein, and the proliferation of virus was inhibited by SPOCK2 protein.

### Example 4. Measurement of Effect of Inhibiting Invasion of Influenza a Virus

The intracellular location of influenza virus when infected by influenza A virus was confirmed by using an immunofluorescent staining.

After labeling V5 to SPOCK2 of Example 3-1 and overexpressing SPOCK2, the A549 cell line was infected with the influenza virus 20TCID50/ml. After culturing the A549 cell line on a cover glass, the cell was immobilized with 4% paraformaldehyde. After culturing at 4° C. for 90 min, the immobilized cell was defined as the virus attachment state, and after culturing at 37° C. for 20 min, the immobilized cell was defined as the initial virus invasion state.

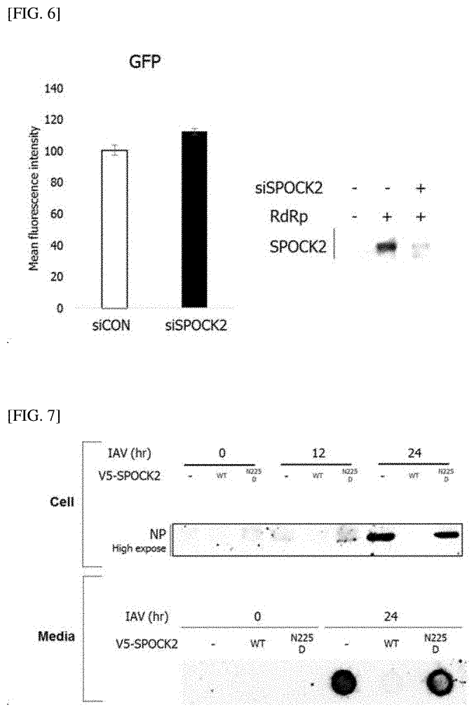

In each state, the penetrability of cell was increased by using 0.2% Triton X-100, and the intracellular location was labeled by using NP (Santacurz), SPOCK2 (Santacruz), Anti-mouse IgG-Alexa-488 (Invitrogen), and Anti-mosue IgG-Alexa568 (Invitrogen) antibodies. For the nucleus of cell, Hoechst 33258 (Sigam) was used. The fluorescent signal was confirmed with a fluorescent microscope and it was analyzed with Image J program, and the result was shown in FIG. 6.

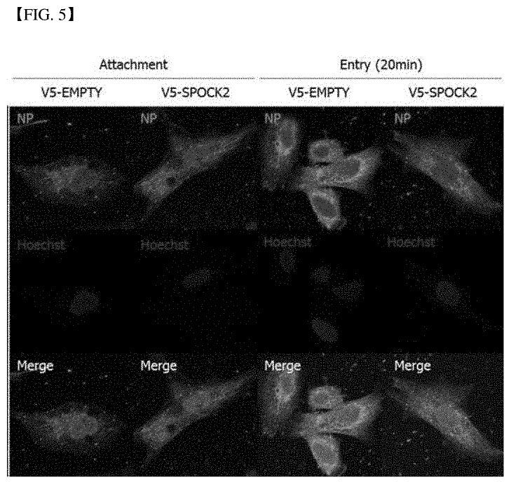

As can be seen in FIG. 5, in case of virus attachment, the location or brightness of NP protein shown as green fluorescence was not changed by overexpression of SPOCK2. However, in the initial process of virus invasion, the brightness of green fluorescence in the cell with overexpressed SPOCK2 was significantly reduced, and thus it was confirmed that SPOCK2 inhibited the virus invasion.

### Example 5. Measurement of Virus Dependent RNA Polymerase Activity Control

To measure the RNA dependent RNA polymerase activity of influenza virus in the A549 cell, after constructing plasmid DNAs of their major factors (PB1, PB2. PA, NP), they were injected in the cell by a liposome introduction method and overexpressed, and the polymerase activity was measured by using the RNA reproduced from them (SEQ ID NO: 11) as a reporter, by using Real Time-qPCR by the same method with the Example 3.

As a result, as can be seen in FIG. 6, it was confirmed that the RNA dependent RNA polymerase activity was not changed by overexpression of SPOCK2.

### Example 6. Confirmation of Effect for Inhibiting Virus Release

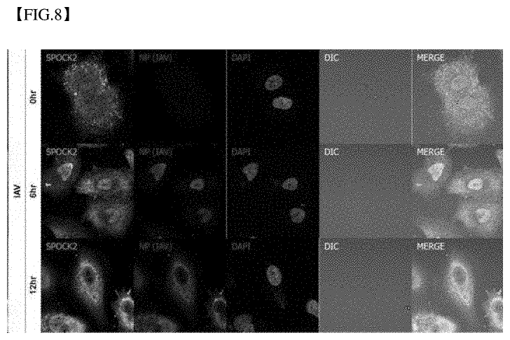

After labeling SPOCK2 of Example 3-1 with V5 and overexpressing SPOCK2, the influenza virus 20TCID⁵⁰/ml was infected to the A549 cell line. Then, to measure the amount of virus released to the outside of the cell, the media in which the cell was cultured were collected and the protein was extracted from them using TCA precipitation. For this, after adding 100% TCA so that the final concentration was 20%, it was reacted on ice for 1 hour. Then, the protein was precipitated through centrifugation at 13000 rpm for 10 minutes and precipitated protein was washed three times with 0.01 M HCl/90% acetone. After isolating the protein on SDS-PAGE through a western blot, the amount of virus was measured by measuring the amount of NP protein using an NP antibody (abcam, ab128193). The result was shown in FIG. 7.

As can be seen in FIG. 7, it was confirmed that the amount of virus released to the media by overexpression of SPOCK2 was significantly decreased. Considering that this phenomenon was not occurred when SPOCK2 N225D-V5 lacking glycosyltaion was used, it can be seen that the glycosylation is important for the effect of virus inhibition of SPOCK2.

### Example 7. Intracellular Location Movement of SPOCK2 According to Influenza Virus Infection

After labeling SPOCK2 of Example 3-1 with V5 and overexpressing SPOCK2, the influenza virus 20TCID⁵⁰/ml was infected to the A549 cell line. After labeling the SPCOK2 and NP protein by an immunofluorescence staining method immediately after infection, in 6 hours, 12 hours, respectively, they were confirmed with a fluorescent microscope.

The specific labeling method was performed as same as the Example 5, and the fluorescence signals confirmed with the fluorescent microscope were shown in FIG. 9

As can be seen in FIG. 8, it was confirmed that the expression of SPOCK2 present near the cell membrane was moved near the nucleus according to the virus infection, and this movement corresponded to the movement of NP protein. Through this, the possibility of controlling the virus infection by co-localization of SPOCK2 and NP was confirmed.

1. A method of inhibiting invasion, release or proliferation of influenza A virus, comprising
preparing a vector containing a gene encoding SPOCK2 protein or a cell transformed by the vector, and
administering the vector or the cell to a subject,

2. The method according to claim 1, wherein the vector is a viral vector.

3. The method according to claim 2, wherein the viral vector is adenovirus, adeno-associated virus, helper-dependent adenovirus or retrovirus vectors.

4. The method according to claim 1, wherein the gene is a nucleotide sequence coding SPOCK2 protein consisting of amino acid sequence of SEQ ID NO: 1.

5. The method according to claim 2, wherein the gene is a nucleotide sequence of SEQ ID NO: 3.

6. The method according to claim 1, wherein the vector has a cleavage map of FIG. 1.

7. The method according to claim 1, wherein the inhibition of influenza A virus proliferation is one or more selected from the group consisting of:
inhibition of intracellular invasion of virus;
inhibition of expression of nucleoprotein (NP) protein of virus; and
inhibition of expression of hemagglutinin (HA) protein of virus.
